(19) United States
(12) Patent Application Publication
(10) Pub. No.: US 2025/0235347 A1
(43) Pub. Date: Jul. 24, 2025

(54) SEMICONDUCTOR COOLING AND HEATING DEVICE

(71) Applicant: Xiamen Ubest Electronic Technology Co., Ltd., Xiamen (CN)

(72) Inventor: Kunlin CHEN, Xiamen (CN)

(73) Assignee: Xiamen Ubest Electronic Technology Co., Ltd., Xiamen (CN)

(21) Appl. No.: 18/655,637

(22) Filed: May 6, 2024

(30) Foreign Application Priority Data
Jan. 22, 2024 (CN) 202420155004.6

(51) Int. Cl. A61F 7/08; A61F 7/00; H01L 23/467

(52) U.S. Cl. CPC A61F 7/08; A61F 2007/003; A61F 2007/0042; H01L 23/467

(57) ABSTRACT

A semiconductor cooling and heating device includes a mounting housing, a cooling and heating semiconductor, and contact pieces. The cooling and heating semiconductor is arranged in the mounting housing, a plurality of contact pieces for contacting with a nursing part are movably connected with the mounting housing along a peripheral surface of the mounting housing, and one end of each contact piece abuts against the cooling and heating semiconductor. The cooling and heating semiconductor enables temperature control and cooling and heating output; the abutment of the contact pieces and the cooling and heating semiconductor enables a quick transfer of temperature to the nursing part; the plurality of contact pieces provided on the peripheral surface of the same mounting housing can provide nursing for multiple parts, and compared with the conventional large-volume and bulky form of separately set nursing components for multiple parts.

### CROSS-REFERENCE TO RELATED APPLICATIONS

This application claims priority to Chinese Patent Application No. 202420155004.6, filed on Jan. 22, 2024, the content of which is incorporated herein by reference in its entirety.

### TECHNICAL FIELD

The present disclosure relates to the field of medical equipment, and in particular, to a semiconductor cooling and heating device.

### BACKGROUND

Currently, the nursing equipment used for knees, shoulders or other joint parts adopts semiconductor cooling and heating controls that are set independently for nursing parts. When there is a nursing need of multiple points, a group of devices are generally set for each point for cooling and heating control. With so many separate devices, the combined nursing equipment is bulky, cumbersome and inconvenient to carry.

### SUMMARY

The technical problem to be solved by the present disclosure is to provide a semiconductor cooling and heating device that is compact in size and capable of meeting the need of cooling and heating control for multiple points.

In order to solve the above technical problem, the present disclosure adopts the following technical solution: a semiconductor cooling and heating device, includes a mounting housing, a cooling and heating semiconductor, and contact pieces, the cooling and heating semiconductor is arranged in the mounting housing, a plurality of contact pieces for contacting with a nursing part are movably connected with the mounting housing along a peripheral surface of the mounting housing, and one end of each contact piece abuts against the cooling and heating semiconductor.

Further, a surface of the contact piece opposite to the nursing part is an arc-shaped surface.

Further, one end of the contact piece is hinged to the mounting housing.

Further, a torsion spring structure is provided between the contact piece and the mounting housing.

Further, the semiconductor cooling and heating device further includes a thermal conductive block, the thermal conductive block is arranged in the mounting housing and abuts against the cooling and heating semiconductor, and one end of the contact piece abuts against the thermal conductive block.

Further, the mounting housing includes a mounting base, the thermal conductive block is arranged on the mounting base, the mounting base is provided with a plurality of fixing seats corresponding to the contact pieces, and the thermal conductive block abuts against the fixing seats; and the fixing seat is provided with a fixing groove, and one end of the contact piece is arranged in the fixing groove.

Further, the mounting housing further includes a pre-pressing block, and the pre-pressing block is arranged above the fixing groove and abuts against one end of the contact piece.

Further, the mounting housing includes an upper mounting housing and a lower mounting housing, the cooling and heating semiconductor is arranged in the upper mounting housing, and the contact pieces are arranged at the lower mounting housing; the upper mounting housing is in bolted connection with the lower mounting housing; and a pressure gap is provided between the upper mounting housing and the lower mounting housing.

Further, the semiconductor cooling and heating device further includes a heat dissipation fan and a heat sink, and the heat dissipation fan and the heat sink are both arranged in the mounting housing; the heat sink and the heat dissipation fan are sequentially arranged on an upper side of the cooling and heating semiconductor; the heat sink abuts against the cooling and heating semiconductor; and the mounting housing is provided with a heat dissipation hole corresponding to the heat dissipation fan.

Further, the semiconductor cooling and heating device further includes infrared lamps, and a plurality of infrared lamps for irradiating the nursing part are provided at intervals along the peripheral surface of the mounting housing.

The semiconductor cooling and heating device of the present application has the following beneficial effects: the cooling and heating semiconductor enables temperature control and cooling and heating output; the abutment of the contact pieces and the cooling and heating semiconductor enables a quick transfer of temperature to the nursing part; the plurality of contact pieces provided on the peripheral surface of the same mounting housing can provide nursing for multiple parts, and compared with the conventional large-volume and bulky form of separately set nursing components for multiple parts, the semiconductor cooling and heating device of the present disclosure is compact and lightweight, and enables multi-point cooling and heating; and the movably connected contact pieces are capable of adjusting a contact angle along with the bending of the nursing part like a knee for better matching.

### BRIEF DESCRIPTION OF THE DRAWINGS

FIG. 1 is a schematic diagram of a semiconductor cooling and heating device according to an embodiment of the present disclosure.

FIG. 2 is an exploded view of a semiconductor cooling and heating device according to an embodiment of the present disclosure.

FIG. 3 is a schematic diagram illustrating cooperation of an upper cloth sleeve pressing gland, a cooling and heating semiconductor, a circuit board and an ambient light of a semiconductor cooling and heating device according to an embodiment of the present disclosure.

FIG. 4 is a schematic diagram illustrating cooperation of a lower cloth sleeve pressing gland, contact pieces and infrared lamps of a semiconductor cooling and heating device according to an embodiment of the present disclosure.

FIG. 5 is a schematic diagram illustrating cooperation of a mounting base and a thermal conductive block of a semiconductor cooling and heating device according to an embodiment of the present disclosure.

### DETAILED DESCRIPTION OF THE EMBODIMENTS

In order to make the technical solutions, objects and effects of the present disclosure clearer, the present disclosure is described in detailed below with reference to the drawings and embodiments.

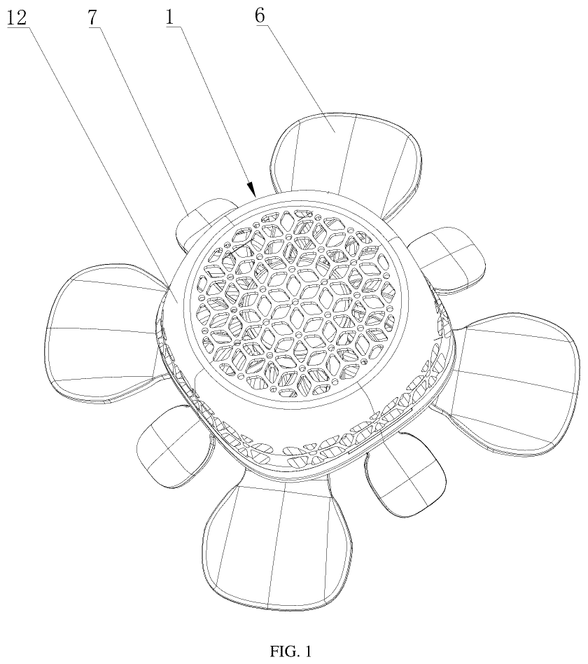

Referring to FIGS. 1 to 5, a semiconductor cooling and heating device includes a mounting housing 1, a cooling and heating semiconductor 2, and contact pieces 6. The cooling and heating semiconductor 2 is arranged in the mounting housing 1, a plurality of contact pieces 6 for contacting with a nursing part are movably connected with the mounting housing 1 along a peripheral surface of the mounting housing 1, and one end of each contact piece 6 abuts against the cooling and heating semiconductor 2.

As can be seen from the above description, the semiconductor cooling and heating device of the present application has the following beneficial effects: the cooling and heating semiconductor 2 enables temperature control and cooling and heating output; the abutment of the contact piece 6 and the cooling and heating semiconductor 2 enables a quick transfer of temperature to the nursing part; the plurality of contact pieces 6 provided on the peripheral surface of the same mounting housing 1 can provide nursing for multiple parts, and compared with the conventional large-volume and bulky form of separately set nursing component for multiple parts, the semiconductor cooling and heating device of the present disclosure is compact and lightweight, and enables multi-point cooling and heating; and the movably connected contact pieces 6 are capable of adjusting a contact angle along with the bending of the nursing part like a knee for better matching.

Further, a surface of the contact piece 6 opposite to the nursing part is an arc-shaped surface.

As can be seen from the above description, the arc-shaped surface of the contact piece 6 can enlarge the contact area with the nursing part.

Further, one end of the contact piece 6 is hinged to the mounting housing 1.

As can be seen from the above description, the hinged design of the contact pieces 6 and the mounting housing 1 enables the contact pieces 6 to rotate and adjust according to the bending of the nursing part, such as a knee, avoiding the compression on the nursing part caused by a fixed structure.

Further, a torsion spring structure is provided between the contact piece 6 and the mounting housing 1.

As can be seen from the above description, the torsion spring structure enables the contact piece 6 to keep abutting against the nursing part at all times.

Further, the semiconductor cooling and heating device further includes a thermal conductive block 3, the thermal conductive block 3 is provided in the mounting housing 1 and abuts against the cooling and heating semiconductor 2, and one end of the contact piece 6 abuts against the thermal conductive block 3.

As can be seen from the above description, the energy of the cooling and heating semiconductor 2 is conducted by the thermal conductive block 3 to the contact pieces 6.

Further, the mounting housing 1 includes a mounting base 15, the thermal conductive block 3 is arranged on the mounting base 15, the mounting base 15 is provided with a plurality of fixing seats 151 corresponding to the contact pieces 6, and the thermal conductive block 3 abuts against the fixing seats 151; and the fixing seats 151 are provided with fixing grooves 152, and one end of the contact piece 6 is arranged in the fixing groove 152.

As can be seen from the above description, the fixing grooves 152 of the fixing seat 151 on the mounting base 15 enable the installation of the contact pieces 6, and the abutment of the thermal conductive block 3 on the fixing seat 151 enables the energy of the cooling and heating semiconductor 2 to be conducted to the contact pieces 6 through the thermal conductive block 3 and the fixing seats 151.

Further, the mounting housing 1 includes a pre-pressing block 141, and the pre-pressing block 141 is arranged above the fixing groove 152 and abuts against one end of the contact piece 6.

As can be seen from the above description, the pre-pressing block 141 above the fixing groove 152 can ensure that one end of the contact piece 6 always remains in contact with the fixing groove 152 to ensure the transfer of energy with the thermal conductive block 3 and the fixing seat 151.

Further, the mounting housing 1 includes an upper mounting housing and a lower mounting housing, where the cooling and heating semiconductor 2 is arranged in the upper mounting housing, and the contact pieces 6 are arranged at the lower mounting housing; the upper mounting housing is in bolted connection with the lower mounting housing; and a pressure gap is provided between the upper mounting housing and the lower mounting housing.

As can be seen from the above description, the upper mounting housing and the lower mounting housing are quickly connected by bolts, and the pressure gap between the upper mounting housing and the lower mounting housing is provided to cooperate with an external cloth sleeve structure, and the external cloth sleeve structure is pressed between the upper mounting housing and the lower mounting housing, and then the device is fixed to the nursing part such as a knee by connecting components such as hook and loop fastener at both ends of the external cloth sleeve structure.

Further, the semiconductor cooling and heating device further includes a heat dissipation fan 5 and a heat sink 4, and the heat dissipation fan 5 and the heat sink 4 are both arranged in the mounting housing 1; the heat sink 4 and the heat dissipation fan 5 are sequentially arranged on an upper side of the cooling and heating semiconductor 2; the heat sink 4 abuts against the cooling and heating semiconductor 2; and the mounting housing 1 is provided with a heat dissipation hole corresponding to the heat dissipation fan 5.

As can be seen from the above description, the heat dissipation fan 5 and the heat sink 4 enable a heat dissipation of the cooling and heating semiconductor 2, and the heat dissipation hole in the mounting housing 1 ensures a good heat dissipation effect.

Further, the semiconductor cooling and heating device further includes infrared lamps 7, and a plurality of infrared lamps 7 for irradiating the nursing part are provided at intervals along the peripheral surface of the mounting housing 1.

As can be seen from the above description, the plurality of infrared lamps 7 provide an infrared irradiation treatment of the nursing part.

### Embodiment 1

Application scenario: when there is a nursing need of multiple points, a group of devices are generally set for each point for cooling and heating control. With so many separate devices, the combined nursing equipment is bulky, cumbersome and inconvenient to carry.

As shown in FIGS. 1 to 5, the semiconductor cooling and heating device of the present embodiment includes a mounting housing 1, a cooling and heating semiconductor 2, a heat sink 4, a heat dissipation fan 5, a thermal conductive block 3, contact pieces 6, infrared lamps 7, and a circuit board 9.

As shown in FIGS. 1 to 3, the mounting housing 1 includes an upper mounting housing and a lower mounting housing, the upper mounting housing includes a fan cover 12 and an upper cloth sleeve pressing gland 13, the fan cover 12 is provided with a plurality of heat dissipation holes, a lower side of the fan cover 12 is snap-in fitted to the upper cloth sleeve pressing gland 13; the heat dissipation fan 5, the heat sink 4 and the cooling and heating semiconductor 2 are provided from top to bottom in the upper mounting housing, the cooling and heating semiconductor 2 may be a commercially available semiconductor chip that is capable of cooling and heating; a middle part of the upper cloth sleeve pressing gland 13 is provided with an opening, and the cooling and heating semiconductor 2 contacts the thermal conductive block 3 through the opening; the upper cloth sleeve pressing gland is provided with a mounting groove, and the circuit board 9 is arranged in the mounting groove; and an ambient light 8 is provided on one side of the upper cloth sleeve pressing gland, and the ambient light 8 is electrically connected to the circuit board 9.

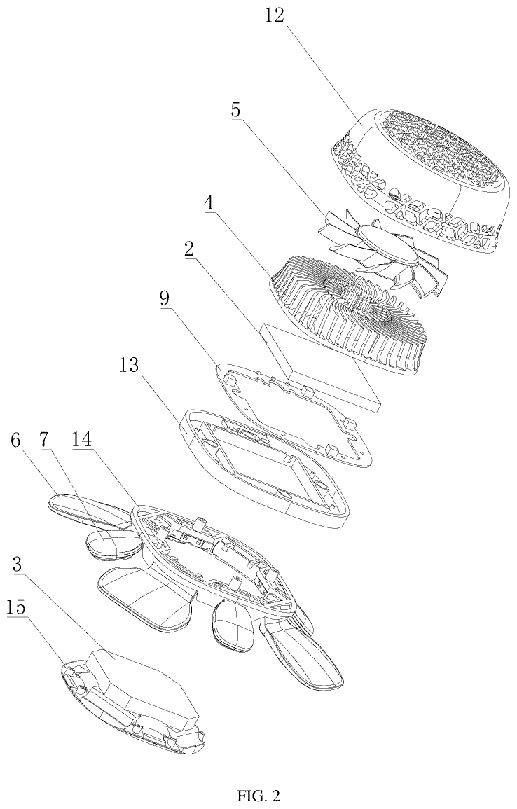

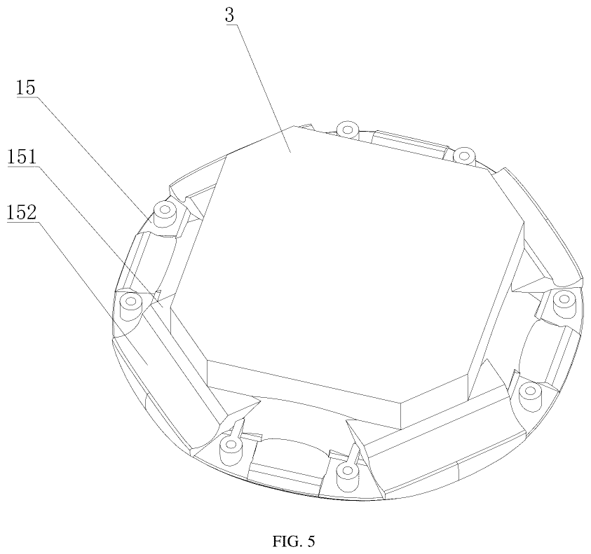

As shown in FIG. 2 and FIG. 5, the lower mounting housing includes a lower cloth sleeve pressing gland 14 and a mounting base 15, the lower cloth sleeve pressing gland 14 is in bolted connecting to the mounting base 15, the thermal conductive block 3 is arranged in a middle part of the mounting base 15, and a middle part of the lower cloth sleeve pressing gland 14 is provided with an opening corresponding to the thermal conductive block 3.

The side, opposite to the nursing part, of the mounting base 15 is concave and has a curved surface structure, which enables a better installation matching with a joint part such as a knee.

As shown in FIG. 2 and FIG. 5, the mounting base 15 is provided with a plurality of fixing seats 151 at intervals along a peripheral surface of the mounting base 15, and the thermal conductive block 3 is correspondingly pressed against the fixing seats 151; the fixing seat 151 is provided with a curved fixing groove 152; the contact piece 6 is cylindrical at one end, and is fan-leaf shaped at the other end, and the side opposite to the nursing part is curved; the cylindrical end of the contact piece 6 is matchedly provided in the curved fixing groove 152, and the contact piece 6 is rotatable relative to the mounting base 15 through the cooperation of the cylindrical end and the curved fixing groove 152. The contact piece 6 is made of thermal conductive metal, preferably made of aluminum alloy. Alternatively, a torsion spring structure is sleeved on the cylindrical end, and one end of the torsion spring structure abuts against the mounting base 15, enabling the contact piece 6 to keep rotatably abutting against the nursing part at all times.

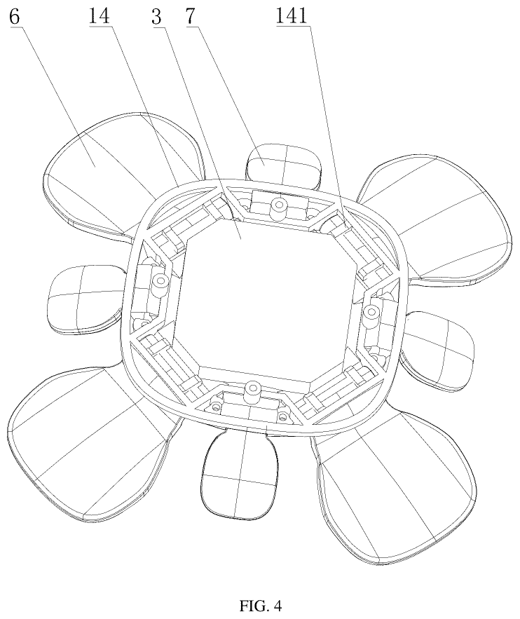

As shown in FIG. 4, a pre-pressing block 141 is provided at the lower cloth sleeve pressing gland 14 above the curved fixing groove 152, and the pre-pressing block 141 presses one end of the contact piece 6 firmly against the curved fixing groove 152.

As shown in FIG. 1, FIG. 2, and FIG. 4, a plurality of infrared lamps 7 are provided at intervals on the peripheral surface of the mounting base 15, and the infrared lamps 7 emit infrared rays towards the nursing part; the infrared lamps 7 are electrically connected to the circuit board 9 at the upper cloth sleeve pressing gland.

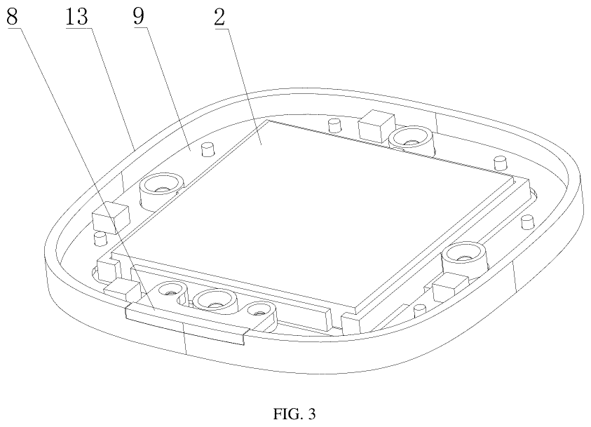

As shown in FIG. 3 and FIG. 4, the lower cloth sleeve pressing gland 14 is provided with a plurality of guide pillars with screw holes, and the upper cloth sleeve pressing gland 13 is provided with connecting holes corresponding to the guide pillars; the insertion matching of the guide pillars and the connection holes enables the lower cloth sleeve pressing gland 14 and the upper cloth sleeve pressing gland 13 to be quickly positioned and matched, and be fixed by locking bolts in the guide pillars.

A pressure gap is formed between the upper cloth sleeve pressing gland 13 and the lower cloth sleeve pressing gland 14, and is used for the clamping installation of the external cloth sleeve structure; and by connecting and fixing the external cloth sleeve structure at the nursing part, the above device can be stably fixed on the nursing part.

Working principle: the device is fixed on the external cloth sleeve structure through the cooperation of the upper cloth sleeve pressing gland 13 and the lower cloth sleeve pressing gland 14, and is fixed at the nursing part such as a knee through connecting components such as a hoop and loop fastener at both ends of the external cloth sleeve structure, and the device is adjusted and controlled through a control box provided on the external cloth sleeve structure.

The temperature is adjusted by the cooling and heating semiconductor 2, and the adjusted temperature is transferred to the contact pieces 6 through the thermal conductive block 3 and the fixing seats 151. As the contact pieces 6 are set at intervals around the peripheral surface of the mounting housing 1, the plurality of contact pieces 6 can cover a plurality of parts, which enables a multi-part cooling and heating control without setting up cooling and heating components separately for each part; the infrared lamps 7 can carry out infrared irradiation therapy on the nursing part, enriching the function of the device; and through the ambient light 8, whether the device is in working condition or not can be judged intuitively.

In summary, the semiconductor cooling and heating device of the present application has the following advantages: the cooling and heating semiconductor enables temperature control and cooling and heating output; the abutment of the contact piece and the cooling and heating semiconductor enables a quick transfer of temperature to the nursing part; the plurality of contact pieces provided on the peripheral surface of the same mounting housing can provide nursing for multiple parts, and compared with the conventional large-volume and bulky form of separately set nursing component for multiple parts, the semiconductor cooling and heating device of the present disclosure is compact and lightweight, and enables multi-point cooling and heating; and the movably connected contact pieces are capable of adjusting a contact angle along with the bending of the nursing part like a knee for better matching.

The above is merely an embodiment of the present disclosure and is not intended to limit the scope of the present disclosure. Any equivalents made within the description and drawings of the present disclosure, or directly or indirectly applying in relevant technical fields, are all included in the scope of the protection of the present disclosure.

What is claimed is:

1. A semiconductor cooling and heating device, comprising a mounting housing, a cooling and heating semiconductor, and contact pieces, wherein the cooling and heating semiconductor is arranged in the mounting housing, a plurality of contact pieces for contacting with a nursing part are movably connected with the mounting housing along a peripheral surface of the mounting housing, and one end of each contact piece abuts against the cooling and heating semiconductor.

2. The semiconductor cooling and heating device according to claim 1, wherein a surface of the contact piece opposite to the nursing part is an arc-shaped surface.

3. The semiconductor cooling and heating device according to claim 1, wherein one end of the contact piece is hinged to the mounting housing.

4. The semiconductor cooling and heating device according to claim 3, wherein a torsion spring structure is provided between the contact piece and the mounting housing.

5. The semiconductor cooling and heating device according to claim 1, further comprising a thermal conductive block, wherein the thermal conductive block is arranged in the mounting housing and abuts against the cooling and heating semiconductor, and one end of the contact piece abuts against the thermal conductive block.

6. The semiconductor cooling and heating device according to claim 5, wherein the mounting housing comprises a mounting base, the thermal conductive block is arranged on the mounting base, the mounting base is provided with a plurality of fixing seats corresponding to the contact pieces, and the thermal conductive block abuts against the fixing seats; and the fixing seat is provided with a fixing groove, and one end of the contact piece is arranged in the fixing groove.

7. The semiconductor cooling and heating device according to claim 6, wherein the mounting housing further comprises a pre-pressing block, and the pre-pressing block is arranged above the fixing groove and abuts against one end of the contact piece.

8. The semiconductor cooling and heating device according to claim 1, wherein the mounting housing comprises an upper mounting housing and a lower mounting housing, the cooling and heating semiconductor is arranged in the upper mounting housing, and the contact pieces are arranged at the lower mounting housing; the upper mounting housing is in bolted connection with the lower mounting housing; and a pressure gap is provided between the upper mounting housing and the lower mounting housing.

9. The semiconductor cooling and heating device according to claim 1, further comprising a heat dissipation fan and a heat sink, wherein the heat dissipation fan and the heat sink are both arranged in the mounting housing; the heat sink and the heat dissipation fan are sequentially arranged on an upper side of the cooling and heating semiconductor; the heat sink abuts against the cooling and heating semiconductor; and the mounting housing is provided with a heat dissipation hole corresponding to the heat dissipation fan.

10. The semiconductor cooling and heating device according to claim 1, further comprising a plurality of infrared lamps for irradiating the nursing part, wherein the plurality of infrared lamps are provided at intervals along the peripheral surface of the mounting housing.
